(19) United States
(12) Patent Application Publication
(10) Pub. No.: US 2003/0009112 A1
(43) Pub. Date: Jan. 9, 2003

(54) Device and electrode arrangement for electrophysiological studies

(76) Inventors: Hugo Hammerle, Tubingen (DE); Wilfried Nisch, Tubingen (DE); Cornelia Leibrock, Wuppertal (DE); Dieter Martin, Hartmannsdor (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/234,664

(22) Filed: Aug. 30, 2002

Related U.S. Application Data
(63) Continuation of application No. PCT/EP01/00728, filed on Jan. 24, 2001.

(30) Foreign Application Priority Data
Mar. 2, 2000 (DE) 100 10 081.3

(51) Int. Cl. A61B 5/05

(52) U.S. Cl. 600/547

(57) ABSTRACT

A device is disclosed for electrophysiological studies on biological material. The device comprises a support on which an array of measurement electrodes is arranged. A vessel is provided having a cavity for the biological material and an appropriate culture medium. The vessel is arranged on the support around the measurement electrodes in such a way that the latter are in electrical contact with the cavity. By means of a counter electrode, which is permanently arranged in the cavity, electrical signals can be measured between the measurement electrodes and the counter electrode.

### CROSS-REFERENCES TO RELATED APPLICATIONS

This application is a continuation of copending international patent application PCT/EP01/00728 filed on Jan. 24, 2001 designating the U.S., which claims priority from German patent application DE 100 10 081.3, filed on Mar. 2, 2000.

### BACKGROUND OF THE INVENTION

The present invention relates to a device for electrophysiological studies on biological material, and in particular to a device having a support on which an array of measurement electrodes is arranged. The device further comprises a vessel having a cavity for the biological material and an appropriate culture medium, with the vessel being arranged on the support and around the measurement electrodes in such a way that the latter are in electrical contact with the cavity, and a counter electrode for measuring electrical signals between the measurement electrodes and the counter electrode, or for electrically stimulating the biological material.

The invention furthermore relates to a microelectrode arrangement for electrophysiological measurements on biological material. Such an electrode arrangement is preferably used in the aforementioned device.

A prior art device and a corresponding electrode arrangement are, for example, disclosed by Egert et al.: A novel organo-typic long-term culture of the rat hippocampus on substrate-integrated multielectrode arrays, Brain Research Protocols 2 (1998), 229-242.

With the known device and the known electrode arrangement it is possible, for example, to study a long-term culture of brain sections or heart muscle tissue. The electrode arrangement is in this case a so-called microelectrode array (MEA) having 60 microelectrodes, which are integrated into a planar substrate. The substrate carries a cylindrical vessel, which is sealed at the bottom by the substrate and, with the latter, forms a cavity in which the array of microelectrodes is arranged.

Biological material, for example tissue with nerve cells and an appropriate culture medium, can be introduced into this cavity in order to incubate cells over a long time. For this purpose, the cylindrical vessel is closed at the top with a lid.

With this device, it is now possible to measure electrical potentials which are generated by the nerve cells when they are active. These potentials result, for example, from changes in the ion concentration inside and outside the cell membrane, and these potential changes can be measured in the vicinity of the nerve cells by electrodes. Of course, electrophysiological studies on any other tissue or cell types are also possible, for example on endothelial cells.

The microelectrode array disclosed by Egert et al., in principle, consists of small titanium nitride (TiN) microelectrodes with a diameter of 10 or 30 μm, and a center spacing of 100 or 200 μm. The microelectrodes are arranged as an array in a culture surface of area 1 cm² on a glass substrate, and they are connected via gold conductor tracks to terminal surfaces outside the array, where contact with a multichannel amplifier takes place. Via the multichannel amplifier, the microelectrodes can be read selectively and the measured signals can be processed further. The principal production process for such microelectrode arrays is disclosed by Egert et al., so that reference is made to this publication for further information.

In principle, the microelectrodes may be produced from various materials; U.S. Pat. No. 5,810,725, for example, discloses a microelectrode array in which the electrode surfaces that come into contact with the culture medium are coated with platinum. Platinum in the form of a thin wire is also used as the counter electrode in this document.

Planar platinum has the disadvantage, however, that a very poor signal-to-noise ratio is encountered with the very small measurement signals. Therefore, in the publication by Egert et al., a method is described for producing a columnar titanium nitride as a material for the microelectrodes, which leads to a significantly better signal-to-noise ratio. This is due to the morphology of the TiN electrodes, which are each formed by thousands of microcolumns having roughly equal diameters of about 0.1 μm and a homogeneous height. This microstructure drastically increases the effective surface area of the electrode, and it consequently reduces the impedance by about an order of magnitude compared with the impedance of flat gold electrodes. A further advantage of TiN electrodes is the mechanical stability, which is very much greater than in the case of electroplated materials, for example platinum. A further advantage is that TiN can be produced in thin film processes, and it is therefore more economical than electroplated materials.

The counter electrode used by Egert et al. is a silver wire at whose lower free end there is a small pressed cylinder of silver chloride. This Ag—AgCl counter electrode permits a significantly better signal-to-noise ratio than when a platinum counter electrode is used. However, the Ag—AgCl counter electrode entails the disadvantage than the silver ions released into the culture medium are toxic to proteins, so that the Ag—AgCl counter electrode can be immersed only temporarily into the culture medium in order to carry out measurements.

The device described so far, comprising a microelectrode array, a vessel and a lid, with the biological material and culture medium contained therein, can be incubated in the usual way, for example in an incubator. For measurement, this device is fitted into a multichannel amplifier, which has appropriate terminal facilities for making contact with the terminal surfaces on the support, so that the measurement amplifiers are connected to the individual measurement electrodes in the cavity. The cover is now removed from the vessel and the Ag—AgCl counter electrode is immersed into the culture medium, and the other end is likewise connected to the measurement amplifier.

On the individual channels, it is now possible to measure the potential differences between the counter electrode and the respective measurement electrode; stimulation of the biological material via chosen measurement channels is also possible. After the measurement, the counter electrode is removed and the lid is replaced in order to continue the cultivation. In this way, a long-term culture of up to four weeks can be maintained and electrophysiologically monitored at regular intervals.

In this case, it is possible to assign the measured activities to particular regions of the tissue sample by also optically recording these. Comparison of the electrophysiological and optical measurement values then allows conclusions about the activities of selected tissue structures and hence the study, for example, of the long-term effect of pharmaceuticals or particular pathophysiological conditions, for example epilepsy or ischemia. Other electrophysiological measurements on biological material can also be carried out. The device described so far, and the electrode arrangement used in it, can be employed for a wide variety of tasks.

However, the inventors of the present application have discovered that the known device suffers from a substantial number of disadvantages which are related, on the one hand, to the fact that in order to carry out the electrophysiological measurements, the lid needs to be removed from the vessel before the thin silver wire with the Ag—AgCl counter electrode can be inserted.

A serious disadvantage in this case involves the handling required: total loss of the sample may occur during the removal of the lid if the person entrusted with the measurement is not careful when taking off the lid and/or transporting the vessel. A further major disadvantage is that the absolutely necessary sterility in the interior of the cavity cannot be guaranteed to a sufficient extent if the lid needs to be taken off repeatedly for the measurements. Contaminants may in this case enter the culture medium not only via the counter electrode, which needs to be immersed repeatedly, but also by the air, or by careless handling.

A further disadvantage is connected with the thin silver wire which, on the one hand, cannot be introduced reproducibly into the culture medium, and this has a disadvantageous effect on the reproducibility of the measurement results between various measurement procedures, since the field profile between the counter electrode and the individual measurement electrodes is also influenced by the position of the counter electrode in the culture medium.

A further disadvantage involves the fact that the Ag—AgCl counter electrode is not only very expensive, but is also highly susceptible to breakage, so that the counter electrode needs to be replaced repeatedly within a series of measurements. This also has a disadvantageous effect on the reproducibility within a monitoring of a long-term culture.

Furthermore, the introduction of the counter electrode into the culture medium entails the risk that the cells of the tissue to be studied may become damaged because the counter electrode has been introduced too far.

Finally, interference can also be coupled in via the silver wire, and this has a particularly disadvantageous effect if the silver wire is moved during a measurement, so that the degree of input coupling changes and/or the position of the counter electrode in the culture medium shifts. Such incidents may be reflected in unallocatable peaks in some or all of the channels.

### SUMMARY OF THE INVENTION

In view of the above, it is an object of the present invention to provide an improved device and an improved microelectrode arrangement such that the aforementioned disadvantages are avoided and, in particular, a more reliable recording is achieved.

It is another object of the invention to provide an improved device and an improved microelectrode arrangement such that a long term study of biological material can easily be achieved.

It is another object of the invention to provide an improved device and an improved microelectrode arrangement such that contamination of the biological material to be studied is easier avoided.

These and other objects are achieved according to one aspect of the invention by the feature that a counter electrode is permanently arranged in the cavity.

According to another aspect, one counter electrode is arranged on a support together with the measurement electrodes.

As the inventors of the present application have discovered, it is not absolutely necessary to immerse a counter electrode into the culture medium only at the individual measurement instants, but rather it can remain so to speak permanently in the culture medium.

In a first exemplary embodiment, the counter electrode is arranged internally on a lid for the vessel.

The counter electrode is in this case carried, for example, on a small projection on the inside of the lid, so that this projection is immersed in the culture medium when the lid is put onto the vessel. The counter electrode is connected, for example, by a thin gold wire to the outside of the lid, where there is a facility for connection to the measurement amplifier.

The counter electrode may in this case consist of conventional materials, for example platinum, which is applied in a known way to the projection.

In this way, it is no longer necessary to open the lid in order to carry out the measurement, so that culture loss or loss of sterility no longer needs to be tolerated.

In a further exemplary embodiment, it is preferred for the counter electrode to be arranged internally on a circumferential wall of the vessel.

This also ensures that the counter electrode is permanently in contact with the culture medium; it may, for example, be applied as an internally circumferential ring onto the cylindrical inner surface of the vessel. As in the case of the counter electrode fitted internally to the lid, contact may be made with the counter electrode on the inner wall of the vessel by means of, for example, a gold wire to the outside, where it is provided with a facility for connection to the measurement amplifier.

In relation to the counter electrode fitted internally to the lid, the further advantage is obtained here in that the culture may be monitored even when the lid is open, if, for example, an optical analysis which cannot be performed through a window provided in the lid is being carried out in parallel.

In a third exemplary embodiment, it is preferred for the counter electrode to be arranged on the support.

This measure is very surprising since, even though the counter electrode now lies so to speak in the plane of the measurement electrodes, good monitoring of the potentials between the counter electrode and the measurement electrodes is nevertheless possible. Until now, it has been assumed in the prior art that the counter electrode should be immersed as centrally as possible from above into the culture medium, as may be the case in the above exemplary embodiment 1 and is described, for example, in Egert et al. and in U.S. Pat. No. 5,810,725 which was mentioned initially. The counter electrode arranged internally on the circumferential wall of the vessel, according to the second exemplary embodiment, also guarantees a very symmetrical field distribution between the counter electrode and the measurement electrodes. For the counter electrode arranged on the support itself, however, it was not to be expected that the field profile would be such that unimpaired measurement of the potentials with corresponding resolution is possible. The inventors of the present application have discovered, however, that this is in fact precisely the case.

The counter electrode may in this case be inserted, for example, laterally through the wall of the vessel and into the cavity. The channel required for this may have a diameter that is so small that no loss of culture medium occurs, and the channel itself may even be sealed after the counter electrode has been inserted.

In a refinement, however, it is preferred for at least one counter electrode to be integrated into the support and, preferably, for it to be produced in the same technology as the measurement electrodes.

These measures have the advantage that one or even several counter electrodes can be produced in a particularly inexpensive way so to speak together with the measurement electrodes. A further advantage is that the measurement electrodes and counter electrode(s) are arranged in such a way that they can be connected to the measurement amplifier in one working step. After the support with the integrated measurement electrodes, as well as the integrated counter electrode, has been produced, the vessel then merely needs to be arranged appropriately on the support, and further handling steps are not required. The counter electrode(s) may in this case be fabricated from materials which have a high effective surface area, for example iridium/iridium oxide.

With the exemplary embodiments described so far, a non-invasive electrophysiological measurement on biological material is possible over a prolonged time, and, because of the at least one counter electrode arranged fixed in the interior of the cavity, the handling is very simple and it has been possible to significantly improve the reproducibility between the individual measurement operations compared with the prior art. Furthermore, the problem of culture losses or contamination is avoided.

In general, it is in this case preferred for the counter electrode to be fabricated from fractal material with microporous structures, for example titanium nitride (TiN), iridium or iridium oxide.

With this measure, it is advantageous that the effective surface area of the counter electrode is increased approximately by two orders of magnitude compared with the area covered internally on the lid, internally on the vessel wall, or on the support, and this is accompanied by a reduction in the impedance by at least approximately an order of magnitude. For this reason, the signal-to-noise ratio with a TiN counter electrode is better by at least a factor of 10 than with a planar gold or platinum counter electrode covering the same area.

A further advantage with the TiN counter electrode is that, compared with a known electrode arrangement with TiN measurement electrodes, virtually no additional costs are encountered when a TiN counter electrode is additionally integrated into the carrier substrate.

Against this background, the present invention furthermore relates to an electrode arrangement for electrophysiological measurements on biological material, with a support into which an array of measurement electrodes as well as at least one counter electrode are integrated.

The advantages already mentioned above are connected with this counter electrode, and a particularly surprising advantage has been found to be that measurements are possible with a very good signal-to-noise ratio and very high resolution, even though the counter electrode(s) lies/lie quite unusually in the plane of the measurement electrodes.

In this case, it is preferred for the counter electrode to have a base surface area which is at least 10³ times larger than a measurement electrode surface area, the base surface area preferably being between about 0.1 mm² and 1 cm², preferably approximately 10-100 mm².

The inventors of the present invention have discovered that even counter electrode base surface areas of this size are sufficient to be able to carry out unimpaired, high-resolution potential measurements. It is furthermore found that with sizes of this order, noise signals can be coupled in only to negligible extents, and shielding of the array and the counter electrode is possible in a straightforward way.

In this case, it is preferred for the counter electrode to lie in immediate proximity to the array, and, preferably, for it to cover a surface, for example a surface in the shape of a half-moon or in the shape of a wedge, which is matched to the profile of the conductor tracks for making contact with the microelectrodes.

With this measure, it is advantageous that the counter electrode actually does not detrimentally affect the arrangement of the measurement electrodes and the supply lines thereof, but nevertheless lies so close to the measurement surface formed by the array of measurement electrodes that the measurement volume can be kept small. In fact, it is merely necessary for the measurement volume to cover the measurement surface and a certain region into which the counter electrode projects. The shape of the counter electrode may in this case be matched to conductor tracks integrated in the substrate. Compared with a counter electrode fitted on the inner wall of the vessel, this provides the further advantage that the measurement volume can be further restricted, for example by a lid extending as far as the bottom with an appropriate gap for the measurement volume, without impairing the measurement facility. Indeed, with such a design the counter electrode on the inner wall of the vessel would no longer be in contact with the culture medium, so that measurements would be impossible.

If the counter electrode in this case covers a wedge-shaped surface, a further advantage is that the conductor tracks, that is to say the connections of the measurement electrodes to the terminal surfaces lying outside the cavity on the support, are not hindered. This is because the surface area of the counter electrode tapers in a wedge shape toward and onto the measurement surface formed by the measurement electrodes, so that virtually the entire periphery of the measurement surface is available for the feed line to the measurement electrodes.

If a plurality of counter electrodes are arranged on the support, then a large surface area can be produced for the counter electrode, with the described advantages. Nevertheless, a single counter electrode that ensures a large effective surface area is also sufficient, especially when it consists of fractal material.

Advantageously, the counter electrode is also electrically connected to a terminal surface on the support outside the cavity.

Against this background, the invention also relates to a method for the electrophysiological study of biological material, in which the novel device and/or the novel electrode arrangement are used.

Further advantages are given in the description and the appended drawing.

It should be understood that the features stated above, and those yet to be explained below, may be used not only in the respectively indicated combinations, but also in other combinations or in isolation, without departing from the scope of the present invention.

### BRIEF DESCRIPTION OF THE DRAWINGS

Exemplary embodiments of the invention are represented in the drawing, and they will be explained in more detail in the description below. In the drawings:

FIG. 1 shows a schematic sectional side view of a device for electrophysiological studies on biological material, in which counter electrodes are arranged internally on a circumferential wall of a vessel and on an inner side of a lid;

FIG. 2 shows a representation as in FIG. 1, in which a counter electrode is arranged internally in the vessel on the support; and

FIG. 3 shows a plan view of the electrode arrangement in FIG. 2.

### DETAILED DESCRIPTION OF PREFERRED EMBODIMENTS

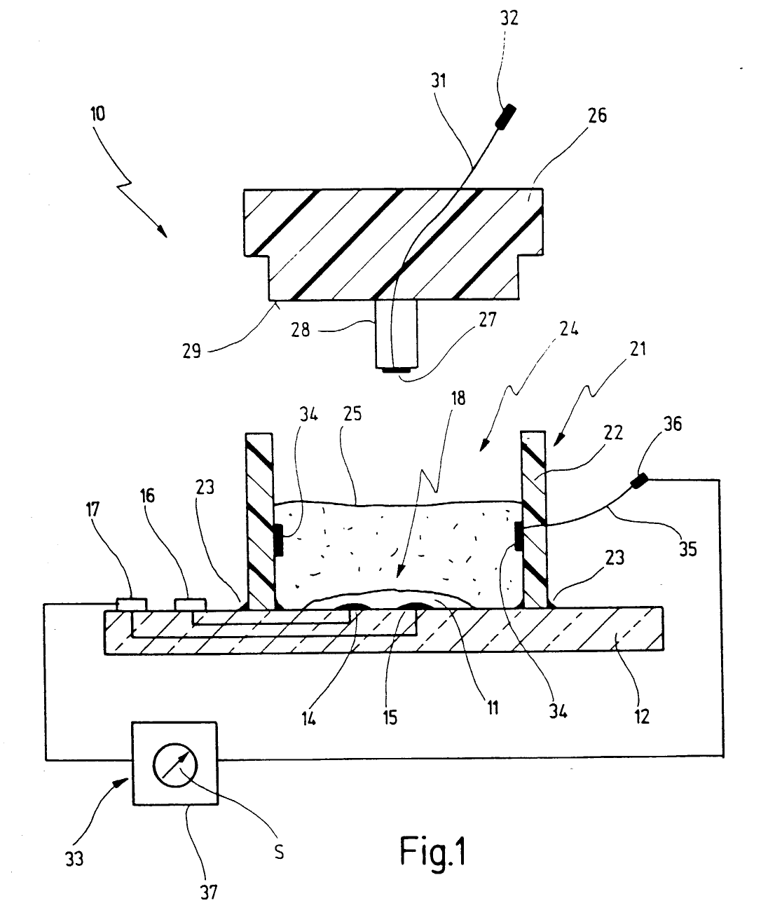

FIG. 1 shows, at 10, a device for electrophysiological studies on biological material that is denoted by 11 in the figure.

The device 10 comprises a support 12, for example made of glass, into which measurement electrodes 14, 15 are integrated; the latter are electrically connected to terminal surfaces 16, 17 which are likewise formed on the support 12.

The measurement electrodes 14, 15 form an array 18, referred to as a microelectrode array, as is described for example in Egert et al. loc. cit. For further information, reference is made to this publication.

A cylindrical vessel 21 is arranged on the support 12, and just like the support 12, it is represented in section in FIG. 1. The cylindrical vessel 21 has a circumferential wall 22, which is adhesively bonded underneath at 23 onto the support 12 in a liquid-tight fashion.

Together with the support 12, the cylindrical vessel 21 delimits a cavity 24, which contains the biological material 11 and an appropriate culture medium 25. When the cavity 24 needs to have a greater height, it is possible to use an adapter ring, sealed by O-rings, which is fitted onto the top of the vessel 21.

Above the vessel 21, FIG. 1 also shows a lid 26 which is used for sterile closure of the vessel 21, or of the adapter ring.

Of course, the cavity 24 is firstly cleaned and sterilized, before the adapter ring is optionally fitted on, and the biological material 11 and the culture medium 25 are introduced into the cavity 24. This sterilization may be carried out, for example, by using steam in an autoclave. After the cavity 24 has been filled, the lid 26 is put on so that the biological material can now be incubated for a long time in the culture medium 25.

Via the electrodes 14, 15 that are electrically connected to the cavity 24 and are arranged in it, the electrical potential of the biological material 11 can now be measured at the terminal surfaces 16, 17. To that end, however, it is necessary to provide a counter electrode in order to record the reference potential via the culture medium 25.

Such a counter electrode 27 is arranged on a projection 28, which is formed on an inner side 29 of the lid 26. Via a line 31, the counter electrode 27 is connected to a plug 32, which is used for connection to a multichannel amplifier 33 to which the terminal surfaces 16, 17 can also be connected.

When the lid 26 is fitted onto the vessel 21, the counter electrode 26 is immersed in the culture medium 25, so that potential differences can be measured between the plug 32 and the terminal surfaces 16, 17.

A further, or alternative, counter electrode 34 is arranged internally as a ring electrode on the circumferential wall 22. The counter electrode 34 is also connected via a line 35 to a plug 36 for connection to the multichannel amplifier 33. FIG. 1 shows, as an example, one channel 37 of the multichannel amplifier 33, which is used to measure a signal S of the measurement electrode 15 that indicates the electrical activity of the biological material 11 in the vicinity of this measurement electrode 15.

While it is possible to take measurements with the counter electrode 27 only when the lid 26 is fitted on, the counter electrode 34 also allows measurements when the lid 26 is open. Both counter electrodes 27, 34 provide a very symmetrical field distribution toward the respective measurement electrodes 14, 15. For the sake of completeness, it should also be mentioned that, for example, 60 measurement electrodes are arranged in the microelectrode array 18, these having a diameter between 10 and 30 μm and being fabricated from titanium nitride, as is described for example in Egert et al. loc. cit.

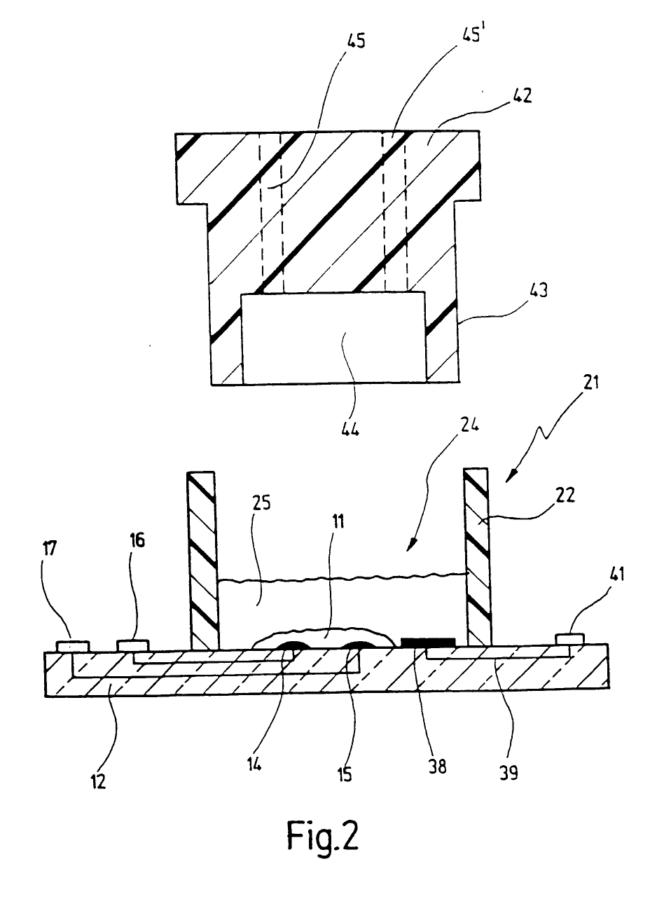

FIG. 2 shows a second exemplary embodiment of the novel device for electrophysiological studies, in which a counter electrode 38 is arranged on the support 12. Like the measurement electrodes 14, 15, the counter electrode 38 is also fabricated from titanium nitride, with the same production method having been used as for the measurement electrodes 14, 15. The counter electrode is connected via a conductor track 39 to a terminal surface 41 outside the cavity 24.

Although the counter electrode 38 is arranged in the plane of the measurement electrodes 14, 15, the arrangement shown in FIG. 2 nevertheless permits reliable measurement of biological material with stable and high-resolution measurement values.

Above the support 12, FIG. 2 shows a lid 42 which has a flange 43 that extends downward onto the support 12. A recess 44 is provided in the lid 42, and it accommodates the measurement electrodes 14, 15, the biological material 11 and a part of the counter electrode 38 when the lid 42 is fitted on. When the lid 42 is inserted, the culture medium 25 in this case collects in the recess 44, so that it is possible to work with a very small measurement volume overall. It is also possible for the culture medium 25 to be introduced into the recess 44, via a closable channel 45 which is indicated by dashes, not until after the lid 42 has been fitted on. A channel 45′ for venting the recess 44 may furthermore be provided.

In such an arrangement, a counter electrode 34 as described in FIG. 1 would be inoperative, since the culture medium 25 could not come into contact with this counter electrode 34.

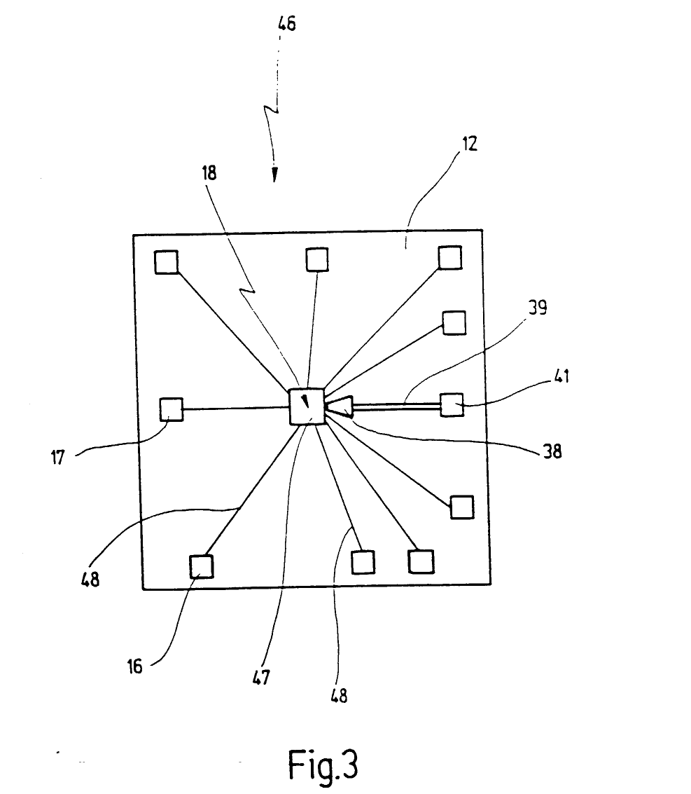

FIG. 3 shows a plan view of a further electrode arrangement 46, as is used for the device 10 in FIG. 2. A measurement surface 47, in which the various measurement electrodes 14, 15 are arranged as an array 18, is indicated centrally on the support 12. Via a schematically indicated conductor track 48, measurement electrodes (not shown in FIG. 3) are connected to terminal surfaces 16, 17 on the support 12. In this way, all four sides of the support 12 are provided with terminal surfaces that lead to particular measurement electrodes 14, 15 inside the measurement surface 47. Laterally next to the measurement surface 47, the counter electrode 38 is shown which covers a wedge-shaped surface that tapers toward and onto the measurement surface 47. In this way, virtually the entire periphery of the measurement surface 47 is available for making contact with measurement electrodes, although because of the wedge-shaped structure of the counter electrode 38, the latter may nevertheless have a size of approximately 20 mm², so that it is larger by more than a factor 10⁴ than the surface of an individual measurement electrode, which here has a diameter of 10 μm.

What is claimed is:

1. A device for electrophysiological studies on biological material contained in an appropriate culture medium, said device comprising:
a vessel forming a cavity for accommodating said biological material and said culture medium,
a support coupled to said vessel and having an array of measurement electrodes arranged on said support, and
one counter electrode permanently arranged on said support,
wherein said vessel is arranged around said measurement electrodes and said one counter electrode in such a way that said measurement electrodes are adapted to make electrical contact with said biologic material, and
wherein said measurement electrodes and said one counter electrode are configured for measuring electrical signals between said measurement electrodes and said one counter electrode, or for providing an electrical stimulating signal to said biological material.

2. The device of claim 1, wherein said one counter electrode has a counter electrode surface area and wherein any of said measurement electrodes have a measurement electrode surface area, said counter electrode surface area being at least 10³ times larger than any of said measurement electrode surface areas.

3. The device of claim 2, wherein said counter electrode surface area is between about 0.1 mm² and 1 cm².

4. The device of claim 3, wherein said counter electrode surface area is between approximately 10 and 100 mm².

5. The device of claim 1, wherein said one counter electrode is located in immediate proximity to said array.

6. The device of claim 1, further comprising a terminal surface for making electrical contact to said one counter electrode, said terminal surface being arranged on said support outside said cavity.

7. A device for electrophysiological studies on biological material contained in an appropriate culture medium, said device comprising:
a vessel forming a cavity for accommodating said biological material and said culture medium,
a support and an array of measurement electrodes being arranged on said support, and
a counter electrode,
wherein said vessel is arranged on said support and around said measurement electrodes in such a way that said measurement electrodes are adapted to make electrical contact with said biological material,
wherein said measurement electrodes and said counter electrode are configured for measuring electrical signals between said measurement electrodes and said counter electrode, or for providing electrical stimulating signals to said biological material, and
wherein said counter electrode is permanently arranged in said cavity.

8. The device of claim 7, wherein said vessel comprises a circumferential wall and said counter electrode is arranged internally of said vessel and on said circumferential wall.

9. The device of claim 7, further comprising a lid for said vessel, said counter electrode being arranged internally of said vessel and on said lid.

10. The device of claim 7, wherein said counter electrode is arranged on said support.

11. The device of claim 10, wherein said counter electrode is integrated into said support.

12. The device of claim 11, wherein said counter electrode is fabricated in the same technology as said measurement electrodes.

13. The device of claim 10, further comprising, for making contact with said measurement electrodes, a plurality of conductor tracks providing a profile, and said counter electrode surface area comprises a shape which is matched to said profile.

14. The device of claim 10, wherein said counter electrode is located in immediate proximity to said array.

15. The device of claim 7, wherein said counter electrode is fabricated from fractal material having microporous structures.

16. The device of claim 15, wherein said counter electrode is made of at least one of the following: titanium nitride, iridium or iridium oxide.

17. The device of claim 7, wherein said counter electrode has a counter electrode surface area and any of said measurement electrodes have a measurement electrode surface area, wherein said counter electrode surface area is at least 10³ times larger than any of said measurement electrode surface areas.

18. The device of claim 17, wherein said counter electrode surface area is between about 0.1 mm² and 1 cm².

19. The device of claim 18, wherein said counter electrode surface area is between approximately 10 and 100 mm².

20. A microelectrode arrangement for electrophysiological measurements on biological material, said electrode arrangement comprising:
a support having an array of measurement electrodes integrated on said support, and
a counter electrode permanently integrated on said support,
wherein said measurement electrodes and said counter electrode are configured for measuring electrical signals between said measurement electrodes and said counter electrode, or for providing an electrical stimulating signal to said biological material.

21. The microelectrode arrangement of claim 20, wherein said counter electrode has a counter electrode surface area and wherein any of said measurement electrodes have a measurement electrode surface area, said counter electrode surface area being at least 10³ times larger than any of said measurement electrode surface areas.

22. The microelectrode arrangement of claim 21, wherein said counter electrode surface area is between about 0.1 mm² and 1 cm².

23. The microelectrode arrangement of claim 22, wherein said counter electrode surface area is between approximately 10 and 100 mm².

24. The microelectrode arrangement of claim 20, wherein said counter electrode is located in immediate proximity to said array.

25. The microelectrode arrangement of claim 20, wherein said counter electrode is fabricated in the same technology as said measurement electrodes.

26. The microelectrode arrangement of claim 20, wherein said counter electrode is made from at least one of the following: titanium nitride, iridium, iridium oxide.

27. The microelectrode arrangement of claim 20, further comprising, for making contact with said measurement electrodes, a plurality of conductor tracks providing a profile, and said counter electrode surface area comprises a shape which is matched to said profile.

28. A method of electrophysiologically studying biological material, said method comprising the steps of
providing a device having a vessel arranged on a support for forming a cavity, an array of microelectrodes being arranged on said support in said cavity, and a counter electrode permanently arranged in said cavity,
introducing a culture medium including said biological material into said cavity, and
measuring electrical signals between said measurement electrodes and said counter electrode.

29. The method of claim 28, further comprising the step of providing electrical stimulating signals to said biological material via at least some of said microelectrodes.
